(19) United States
(12) Patent Application Publication
(10) Pub. No.: US 2009/0098031 A1
(43) Pub. Date: Apr. 16, 2009

(54) Method and apparatus for sanitizing shoe soles

(75) Inventor: Timothy Terrance Crist, Parker, CO (US)

Correspondence Address: REILLY INTELLECTUAL PROPERTY LAW FIRM, 1325 East 16th Avenue, DENVER, CO, 80218, US

(73) Assignee: TC Enterprise

(21) Appl. No.: 11/973,655

(22) Filed: Oct. 10, 2007

(51) Int. Cl. A61L 2/00

(52) U.S. Cl. 422/300; 422/292

(57) ABSTRACT

A shoe sole sanitizing device is made up of a tray having a reservoir containing a sanitizing solution, a perforated grate with scraping edges on the tray being of a resiliency sufficient to be depressed beneath the upper surface of said solution when stepped on by a wearer, and a drain for removal of used solution and particles which collect in the reservoir. In one version, the fill and drain system may be mounted within the tray, or mounted on the tray in a portable version that may be easily moved from place to place, or in another version may have greater capacity for high volume use.

### BACKGROUND AND FIELD

The following relates to footwear and more particularly relates to a novel and improved method and apparatus for sanitizing shoe soles in a rapid, thorough and efficient manner.

Numerous approaches have been taken to the use of cleaning devices for shoes at the entry to homes, businesses or specific areas of a building, which are required to remain free of debris. For example, it has been proposed to utilize a floor tray with a grate, which can be depressed and cause a washing solution to saturate the sole and lower part of the shoe. Another approach utilizes a foot tray with an upstanding wick saturated with a cleaning solution along with an absorbent mat for the wearer to use in drying off each shoe. Still another approach utilizes a separate scraper along with a separate cleansing solution to be used in successive steps in cleaning each shoe.

There is a continuing need for a compact floor-supported sanitizing device which can be conveniently mounted or otherwise supported on a floor surface and so constructed and arranged as to enable simultaneous soaking and scraping of the undersurface and sides of each shoe sole; providing a clean, sanitized shoe surface. This is certainly important in buildings having clean rooms which are required to remain bacteria-free and further which will minimize overflowing or release of the sanitizing solution onto the surrounding area of the device and enable more rapid, thorough sanitizing and drying of each sole. Furthermore, it is desirable to provide an automated mixer and timer system for draining and filling the device.

### SUMMARY OF ADVANTAGES AND FEATURES

In one aspect, it is desirable to provide for a novel and improved shoe sole sanitizing device which enables simultaneous scraping and soaking of the undersurface and sides of a shoe sole in order to prevent tracking of moisture, dust and contaminants into interior living areas of a room or building and in such a way as to prevent overflowing or unwanted discharge of the cleansing or sanitizing solution. In another aspect, it is desirable to provide for an improved shoe sole sanitizing device which can be placed in or on a floor surface, requires a minimum amount of a sanitizing solution, and the solution can be accurately and automatically mixed, metered and drained in separate or built-in holding tanks associated with the sanitizing unit.

The above and other features are carried out in several embodiments of a shoe sole sanitizing device which comprises a tray containing a sanitizing solution, a metal perforated grate releasably positioned on an upper open end of the tray, the grate being of a resiliency sufficient to be depressed a limited distance beneath the upper surface into the solution when downward weight is applied by the wearer of the shoe to soak and remove foreign particles from the undersurface of the shoe sole, and a drain mounted beneath the grate for removal of the solution and particles which have collected in the drain. In order to further encourage the removal of foreign matter from each shoe, the grate has scraping edges extending above the grate top surface, and the drain has downwardly sloping surfaces beneath the grate, which terminate in an outlet for removal of the sanitizing solution. An automatic timer can be associated with a valve in the outlet and with a mixer for automated mixing, filling and draining of the solution to and from the tray.

It is therefore to be understood that even though numerous characteristics and advantages of the present embodiments have been set forth in the foregoing description, together with details of the structure and function of the embodiments, the disclosure is illustrative only, and changes may be made within the principles of the embodiments to the full extent indicated by the broad general meaning of the terms in which the appended claims are expressed and reasonable equivalents thereof.

The above and other advantages and features will become more readily appreciated and understood from a consideration of the following detailed description of different embodiments when taken together with the accompanying drawings in which:

### BRIEF DESCRIPTION OF THE DRAWINGS

FIG. 1 is an exploded view of one embodiment of a shoe sole sanitizing device;

FIG. 2 is a cross-sectional view of the embodiment shown in FIG. 1;

FIG. 3 is a fragmentary sectional view of a tray support taken from FIG. 2;

FIG. 4 is a top plan view of the one embodiment;

FIG. 5 is a top plan view with a grate removed;

FIG. 6 is a cross-sectional view of the one embodiment positioned adjacent to a stairway;

FIG. 7 is a detailed perspective view of a grate employed in the one embodiment;

FIG. 8 is a close-up view of a corner of FIG. 7;

FIG. 9 is a perspective view of a second embodiment of a shoe sole sanitizing device;

FIG. 10 is a cross-sectional view of the second embodiment of FIG. 9;

FIG. 11 is a perspective view of the grate employed in the second embodiment;

FIG. 12 is a perspective view of a third embodiment of a shoe sole sanitizing device;

FIG. 13 is a cross-sectional view of the tray of the third embodiment; and

FIG. 14 is a perspective view of the grate of the third embodiment.

### DETAILED DESCRIPTION OF ONE EMBODIMENT

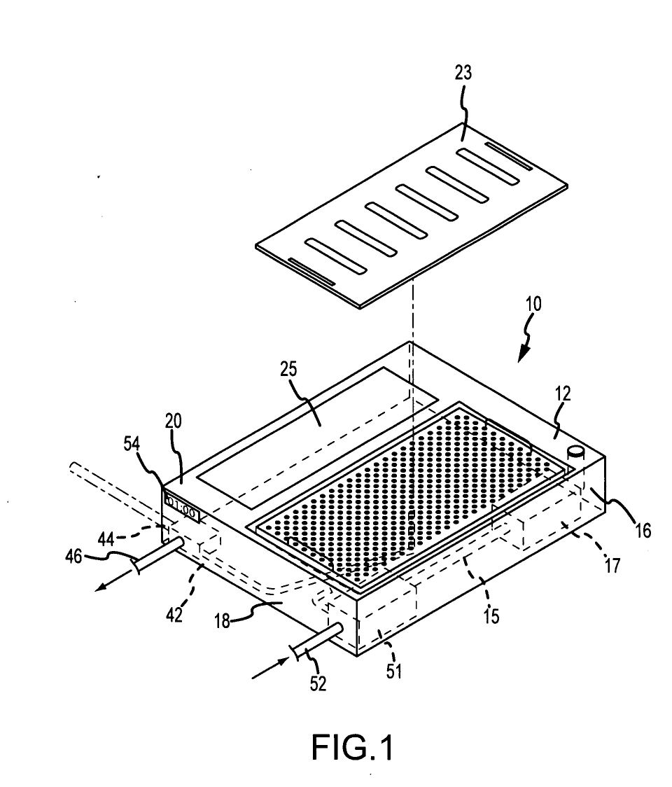

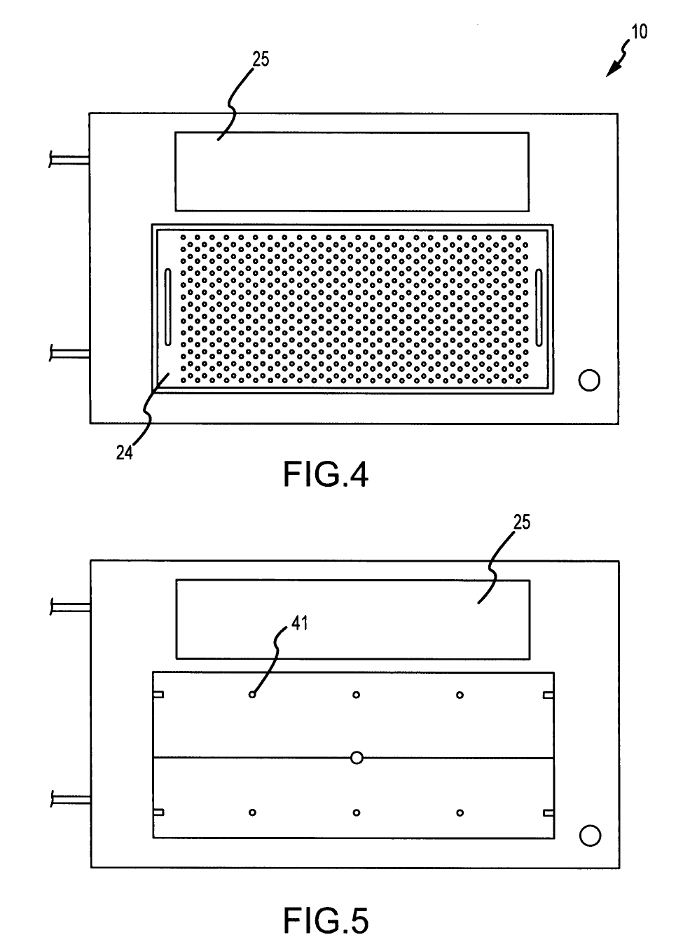

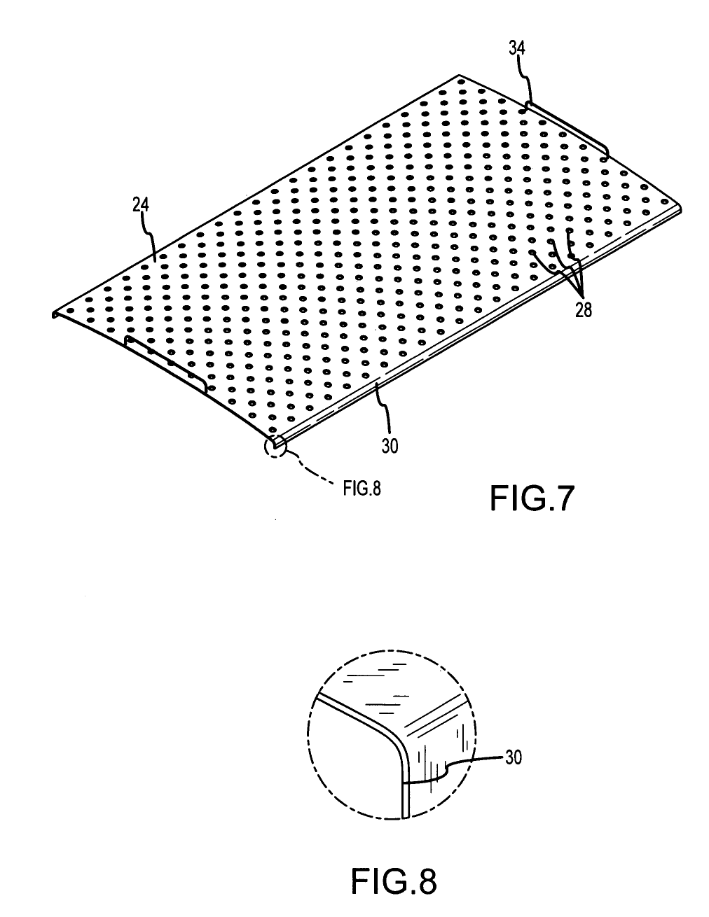

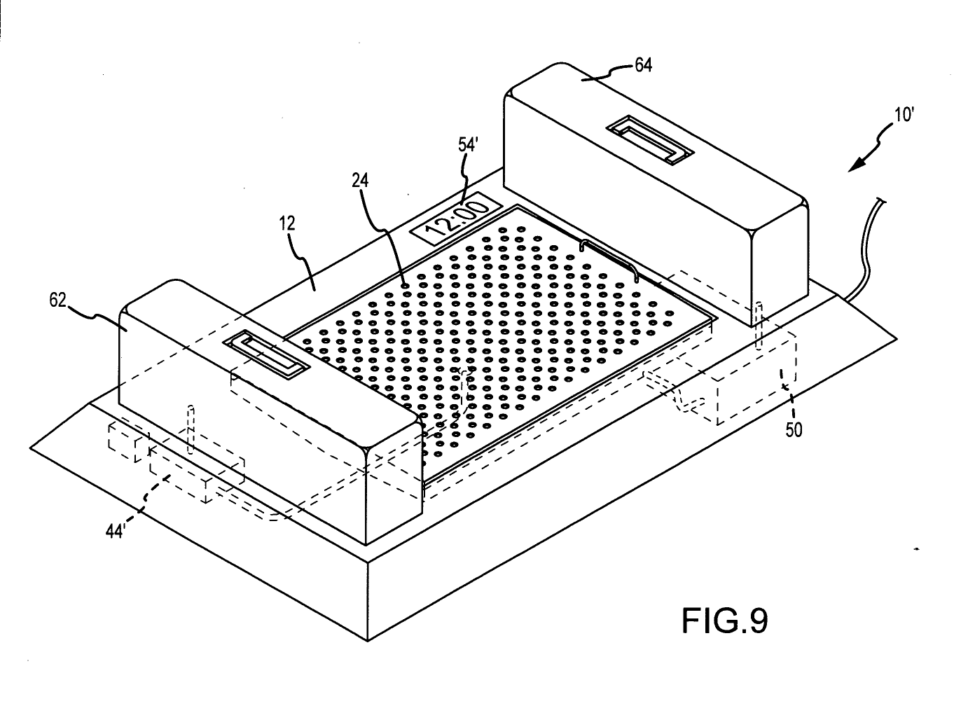

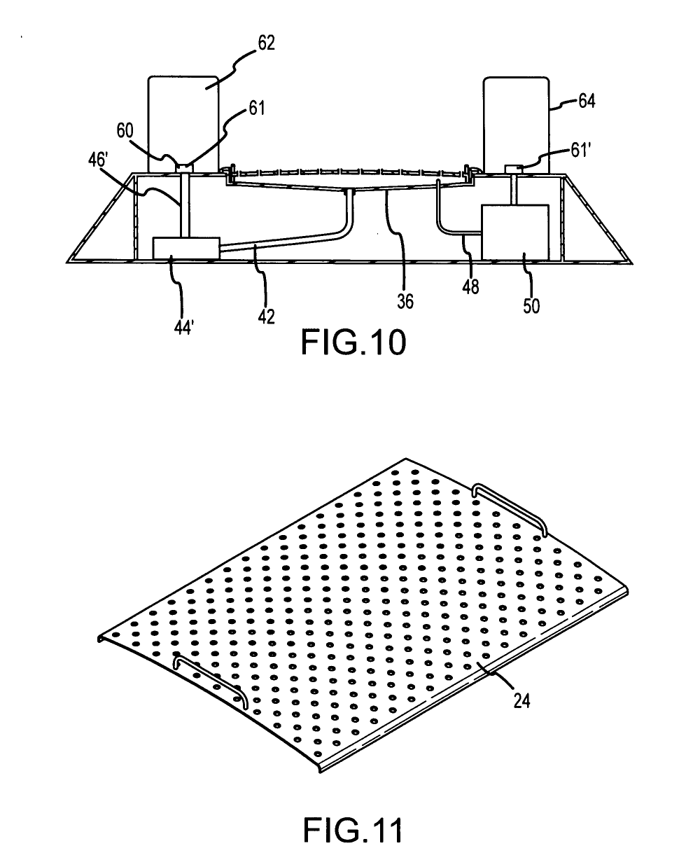

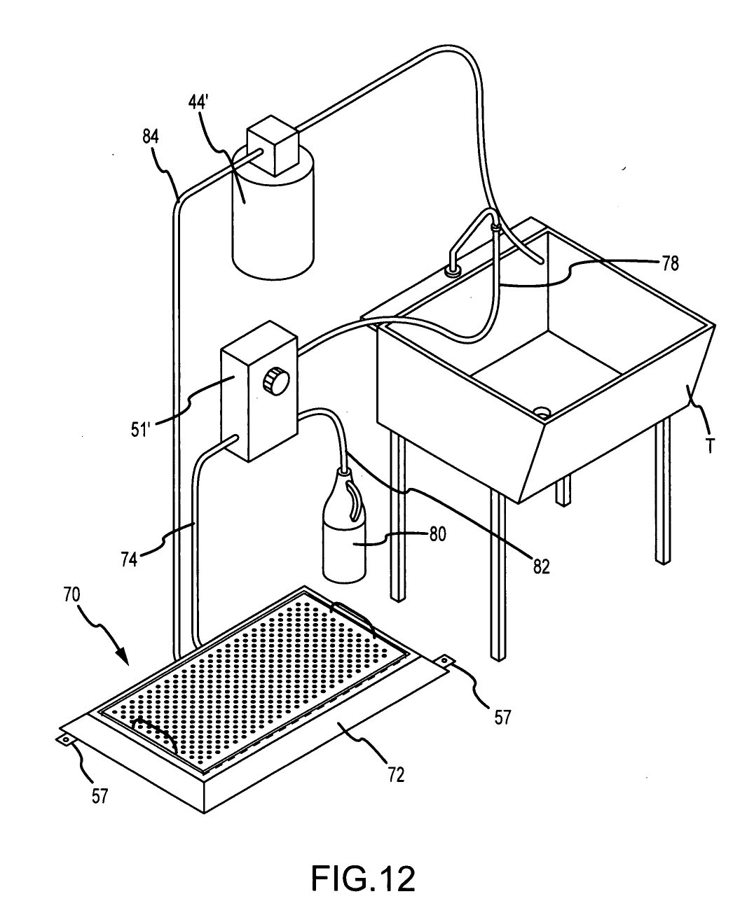

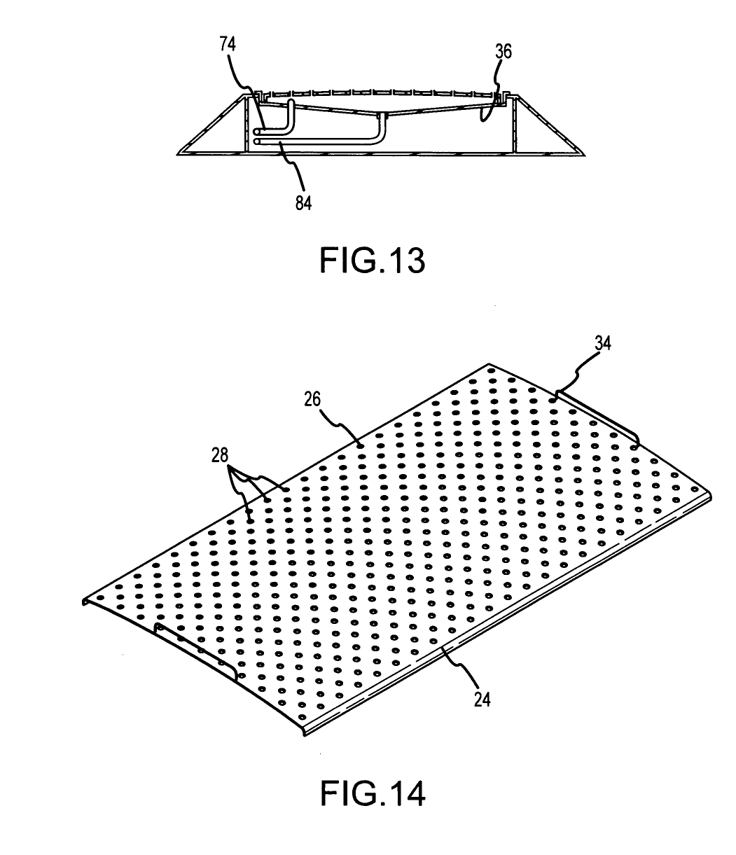

Referring in detail to the drawings, a shoe sole sanitizing device is adapted for use in homes, businesses, medical facilities, processing plants, manufacturing plants and food preparation areas. Referring in detail to the drawings, FIGS. 1 to 8 illustrate one embodiment for use in providing floor surface sanitation to homes, although it is readily conformable for use in other applications. The unit may be placed in the sub-floor and it stops the microorganisms that are collected on the soles of your shoes in public areas from entering the home as well as removing debris. FIGS. 9 to 11 illustrate a second embodiment of shoe sole sanitizing device which is designed more as a portable unit and either may be battery-operated or with a single power cord source and requires no fill or discharge lines. FIGS. 12 to 14 illustrate a third embodiment designed for industrial use or other applications in heavy traffic areas, which require greater solution and pumping capacity and is mountable on an existing surface. Again, the three embodiments hereinafter described are given more as a setting and by way of an illustrative example to illustrate the versatility and efficiency of the system.

Referring to FIGS. 1 to 8, a shoe sole sanitizing device 10 is comprised of a generally rectangular tray 12 having a flat base 14, opposite side walls 16, opposite squared end walls 18 and a flat top panel 20 permanently affixed to the upper edges of the side and end walls 16 and 18. The panel 20 has a grate-receiving opening 22 spanning the greater part of the length and approximately one-half the width of the top panel 20 so that an absorbent mat or rug 25 may be positioned on the top panel 20 in juxtaposed relation to the grate 24. The mat may include a heavy-weight universal mat pad or vinyl loop indoor/outdoor mat that resists mildew.

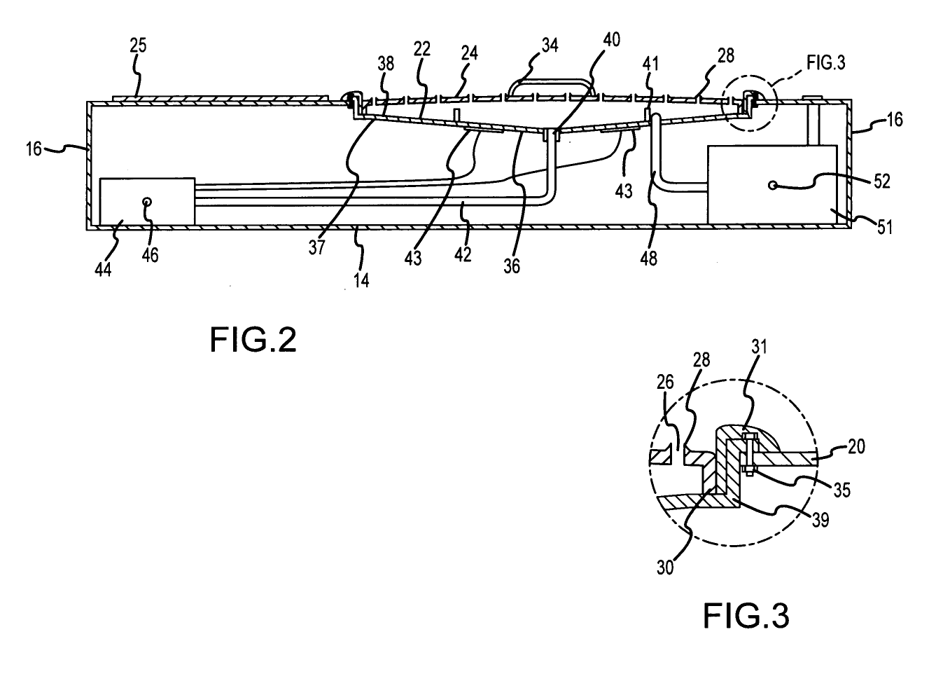

The grate 24 is illustrated in more detail in FIGS. 2, 4, and 7 and is provided with a series of perforations or through-bores 26 at closely spaced intervals throughout the length and width of the grate, although it will be apparent that the perforations simply may be concentrated around the central area. The grate is composed of a metal such as stainless steel or other material which can be formed into a slightly concave configuration so as to be depressed toward the center from opposite ends and is capable of being further depressed downwardly when weight is applied by a wearer in stepping onto the grate. The downward depression of the grate forces sanitizing solution upwardly to penetrate the bores 26 consequently forcing sanitizing solution upwardly into the soles and any treading on the shoe. The upper surface of the grate is provided with scraper edges 28 which may be formed by burred edges around the bores 26 when the bores are punched through the grate to form a raised surface or scraping edge. In addition, opposite sides of the grate are provided with edges 30 which rest against ledges 31 and outer vertical side and end flanges 39 of the opening 22 in the top panel 20 so that the outer edges of the grate will be substantially flush with the top surface 20. The outer flanges 39 are affixed to the surface of the top panel 20, such as, by soldering, welding or use of fasteners 35 that secure the ledges 31 and side flanges 39 to the top surface 20 as shown in FIG. 3. A shallow receptacle or reservoir 36 is suspended from the undersurface of the top panel 20 so as to span or traverse the grate-receiving opening 22 and is provided with sloped walls 38 which converge downwardly from the outer vertical side and end flanges 39 and terminate in a common central opening 40. The outlet 40 has a drain filter (not shown) to prevent large particles from clogging a drain line 42 that extends into drain pump 44. For example, but not by way of limitation, the drain pump 44 may be a KNF Flexible Impeller Pump, No. AC-106-10, manufactured by KNF Neuberger Inc. of Trenton, N.J. A discharge line 46 extends from the pump 44 into a waste collection area or sewer line, not shown. The discharge line 46 may extend from the sidewall 16 or the end wall 18. A fill line 48 extends from a hydro mixer 51 into the receptacle 36. The hydro mixer 51 is a mixing valve that requires the combination of water pressure and use of siphoning to mix a chemical with water to dilute it. One version is Hydro Mixer Hydro Streamline No. 8351 manufactured by Hydro Systems Co. of Cincinnati, Ohio. The hydro mixer 51 has an inlet line 52 for the introduction of water from an outside source and a sanitation line 15 for the introduction of sanitizing solution into the hydro mixer 51. A sanitizing solution receptacle 17 holds sanitation chemical. One commercial sanitation chemical that may be utilized with the Hydro Mixer is Misty Clear Lemon 10 disinfectant manufactured by Amrep Inc. of Marietta, Ga. Another sanitation chemical that may be used is Pro-San® manufactured by Microcide®, Inc. of Detroit, Mich. The sanitizing solution receptacle 17 may be filled by a user with the sanitizing solution or the receptacle may be sold as an insertable, one-time use cartridge having a pre-determined amount of sanitizing solution. Once used, the cartridge may be disposed of and replaced with a new one.

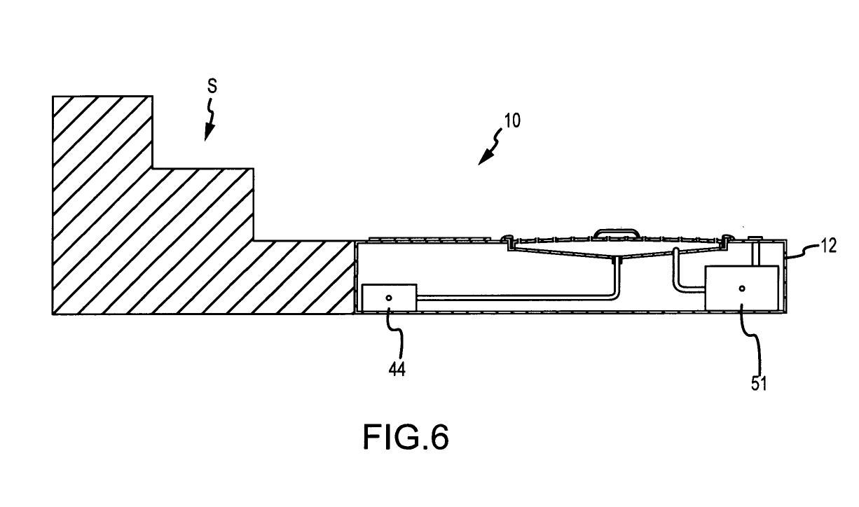

The fill line 48 extends upwardly through one of the sloping walls 38 into communication with the reservoir 36. In addition, one or more limit stops 41 as shown in FIG. 2 and FIG. 5 project upwardly from the sloped walls 38 to limit or regulate the extent of downward movement or depression of the grate when weight is applied. The limit stops 41 may be fixed or adjustable so as to assure that the shoe sole will sink into the solution only along the undersurface and sides of the sole without contacting the rest of the shoe. For the purpose of illustration but not limitation, the grate may be on the order of one-eighth inch thick and would be pressed down so that the solution would rise about one-quarter inch up the side of the shoe, and that height can be adjusted based on the level that the solution is filled to in the base tray. Heating pads 43 may be incorporated and attached along an outside surface 37 of the sloped walls 38, to prevent freezing of the sanitation solution. Offered by way of example and not limitation, the IDP Pad Heater Model 4.5 distributed by Industrial Diesel Products, Inc of Ontario, Canada, may be used as a heat source.

In use, a timer 54, as illustrated in FIG. 1, is automatically activated to turn on the drain pump 44 to drain the solution out through the line 42 and then turn on the hydro mixer 51 to refill the reservoir 36 to the proper level. Typically, for home use, the refill interval may be set for once a day but of course high traffic environments may need this process done more than once a day. A test strip kit, not shown, may be utilized to calibrate the system to determine how frequently it may need to be changed and maintain the sanitation solution at a level that will still break down the microorganisms. For example, the electronic timer 54 may be an HD Digital 7-Day Timer No. 2509 manufactured by SmartHome of Irvine, Calif. An indicator float (not shown) or electronic sensor (not shown) may also be used in the reservoir 36 and receptacle 17 to signal low solution so that additional solution may be added to the reservoir or the receptacle.

When a user or wearer steps on the grate with each shoe and slides or shifts the shoe along the surface, the scraping edges 28 will loosen or dislodge any foreign particles in the tread of the shoe sole while at the same time permitting the solution to thoroughly saturate the tread and undersurface area of the sole. Again, the downward depression of the grate forces sanitizing solution upwardly to penetrate the bores 26 consequently forcing sanitizing solution upwardly into the soles and any treading on the shoe. After each sole is moistened it is then advanced to the mat 25 to remove any excess solution and to accelerate the drying of the shoe sole. The shoe soles may be done singly or in pairs depending to some extent on the size of the shoe. As shown in FIG. 1, the upper surface of the grate may be textured or covered with a sand paper or other gritty material to assist in dislodging solid particles and a separate lid or cover 23 may be placed over the grate area.

FIG. 6 illustrates the manner in which the sanitizing device 10 can be utilized at the bottom of an entry stairway or entry port designated at S. Most desirably, the sanitizing device 10 is dimensioned to be of a height corresponding to the riser dimension of a standard stairway so that its upper surface will essentially be flush with the tread portion of the bottom stair. Although not shown, suitable power cords are provided for extension from the hydro mixer 51 and pump 44 as well as the electronic timer 54 for the unit 10.

### DETAILED DESCRIPTION OF SECOND EMBODIMENT

Referring in detail to FIG. 9 to 11, a second embodiment of a shoe sole sanitizing device 10′ is illustrated and wherein like parts are correspondingly enumerated and comprise once again of a tray 12 without the absorbent mat 26 of FIGS. 1 to 8, and the size and specifically the width of the tray unit is reduced to accommodate a grate 24 and also contains a reservoir 36 with a fill line 48 extending from a filler pump 50. An absorbent matt may accompany the portable unit but is not incorporated into the unit. Again, a drain line 42 extends from the bottom of the reservoir to a pump 44′ which discharges the solution from the reservoir prior to re-filling with fresh solution. A discharge line 46′ extends upwardly into an inlet 60 of a recovery tank 62 for storing waste solution. The inlet 60 is provided with a check valve 61 which automatically opens in response to fluid under pressure directed upwardly through the discharge line 46′. A supply tank 64 is provided with an outlet having a check valve 61′ which will open in response to reduction in pressure from the filler pump 50 in order to pump a sanitizing solution into the reservoir 36. The supply tank 64 may be a one-time use removable sanitation solution cartridge or may be refilled with sanitation solution by the user. The electronic timer 54′ may be an HD Digital 7-Daytimer 2509 manufactured by SmartHome of Irvine, Calif. Again, the sanitizing solution may be Misty Clear Lemon 10 disinfectant manufactured by Amrep Inc. or Pro-San® manufactured by Microcide®, Inc.

### DETAILED DESCRIPTION OF THIRD EMBODIMENT

FIGS. 12-14 illustrates an industrial shoe sole sanitizing device 70 in which like parts to those of FIGS. 1 to 8 are correspondingly enumerated and is broadly comprised of a tray 72 corresponding to the basic construction of the grate portion of the tray of FIGS. 1 to 8 and including a perforated grate 24 removably positioned within a top panel 20 so as to be disposed over a reservoir 36. A fill line 74 is connected between the reservoir 36 and a hydro mixer 51′. The hydro mixer 51′ receives water through a supply line 78 from a conventional sink or laundry tub T or any other water source and a sanitizing solution from a suitable container 80 via supply line 82. The container 80 may be a disposable, one-time use sanitation solution container or be refillable by a user. Similarly, a drain line 84 extends from the reservoir 36 to an externally located drain pump 44″ which drains into the sink or tub T or any other discharge system. In a manner similar to the mobile unit 10′ of FIGS. 9 to 11, the grate 24 is releasably positioned over the reservoir and is provided with closely spaced perforations 26 and upwardly projecting scraper edges 28 as illustrated in more detail in FIGS. 2 and 3. The reservoir 36 is sloped downwardly toward the center, and the sanitizing solution is pumped from the mixer 51′ into the reservoir 36 to fill the reservoir to a pre-determined level in close proximity to the undersurface of the grate 24. The advantages of the tray unit 72 are that it has fewer internal pieces than the tray unit 12 of FIGS. 1 to 8 and can handle a larger volume of solution. Further, the fill and drain line 74 and 84 can be made of the desired length to permit the tray unit to be located at the desired distance away from fill and drain apparatus as described. Also, the tray unit may be placed on an existing surface and secured to the surface with tabs 57.

It is therefore to be understood that even though numerous characteristics and advantages of the present embodiments have been set forth in the foregoing description, together with details of the structure and function of the embodiments, the disclosure is illustrative only, and changes may be made within the principles of the embodiments to the full extent indicated by the broad general meaning of the terms in which the appended claims are expressed and reasonable equivalents thereof.

I claim:

1. A shoe sole sanitizing device comprising in combination:
a tray containing a sanitizing solution therein;
a perforated grate positioned on an upper open end of said tray, said grate being of a resiliency sufficient to be depressed a limited distance beneath the upper surface of said solution when stepped on by a wearer of said shoe sole to soak and remove foreign particles from said lower surface of said shoe sole; and
a drain mounted beneath said grate including means for removal of said solution and particles from said shoe sole, which have collected in said drain.

2. A shoe sole sanitizing device according to 1 wherein said grate is of slightly concave configuration and has perforations with upper scraping edges extending above a top surface of said grate.

3. A shoe sole sanitizing device according to claim 1 wherein said drain includes downwardly sloping wall members converging into an outlet beneath said grate.

4. A shoe sole sanitizing device according to claim 3 wherein said outlet is centrally located within said tray.

5. A shoe sole sanitizing device according to claim 3 wherein said drain includes a check valve associated with said outlet.

6. A shoe sole sanitizing device according to claim 1 wherein an absorbent mat is positioned on said tray in juxtaposed relation to said grate.

7. A shoe sole sanitizing device according to claim 1 wherein said tray includes means for limiting downward movement of said grate in response to being stepped on by the wearer.

8. A shoe sole sanitizing device according to claim 1 wherein said tray includes a cover.

9. A shoe sole sanitizing device according to claim 1 wherein said tray includes a heat source.

10. A shoe sole sanitizing device wherein said tray is adapted to be mounted in a sub-floor.

11. A shoe sole sanitizing device comprising in combination:
a tray containing a sanitizing solution therein;
a metal perforated grate releasably positioned across an upper open end of said tray, said grate being of a resiliency sufficient to be depressed downwardly beneath the upper surface of said solution when stepped on by a wearer of said shoe sole to soak and remove foreign particles from said lower surface of said shoe sole;
a reservoir mounted beneath said grate including means for removal of said solution and particles from said shoe sole which have collected in said reservoir, and a recovery tank removably positioned on said tray in communication with said reservoir; and
a holding tank removably mounted on said tray for storage of said solution, and fill means including a pump between said hold tank and said reservoir for partially filling said reservoir with said solution.

12. A shoe sole sanitizing device according to claim 11 wherein said grate has perforations with upper scraping edges extending above a top surface of said grate.

13. A shoe sole sanitizing device according to claim 11 wherein said drain includes downwardly sloping wall members converging into an outlet beneath said grate, and a check valve associated with said outlet.

14. A shoe sole sanitizing device according to claim 13 wherein said outlet is centrally located within said reservoir and includes a drain filter.

15. A shoe sole sanitizing device according to claim 11 wherein said recovery tank and said holding tank each includes a liquid inlet provided with a check valve.

16. A shoe sole sanitizing device according to claim 11 wherein said reservoir slopes downwardly from opposite ends toward a center, and means provided for limiting downward movement of said grate in response to weight being applied to said grate.

17. A shoe sole sanitizing device according to claim 11 wherein said recovery tank and said holding tank are mounted at opposite ends of said grate on said top panel.

18. A shoe sole sanitizing device comprising in combination:
a tray containing a sanitizing solution therein;
a perforated grate releasably positioned within an upper open end of said tray, said grate being of a resiliency sufficient to be depressed a limited distance beneath the upper surface of said solution when stepped on by a wearer of said shoe sole to soak and remove foreign particles from said lower surface of said shoe sole;
a reservoir suspended beneath said grate including means for removal of said solution and particles from said shoe sole which have collected in said reservoir;
means including a mixer mounted externally of said tray for filling said reservoir; and
means including a discharge pump mounted externally of said tray for draining said solution and particles which have collected in said reservoir.

19. The shoe sole sanitizing device according to claim 18 wherein a fill line extends from said mixer to said reservoir and a water line extends from a water source to said mixer.

20. The shoe sole sanitizing device according to claim 18 wherein said grate is metal and means are provided in said reservoir for limiting the distance of downward movement of said grate when stepped on by a wearer.

21. The shoe sole sanitizing device according to claim 18 wherein downward movement of said grate forces said solution to flow upwardly onto said shoe sole.
